(19) United States
(12) Patent Application Publication
(10) Pub. No.: US 2002/0028754 A1
(43) Pub. Date: Mar. 7, 2002

(54) Antimicrobial compositions

(75) Inventors: Charlotte Johansen, Holte (DK); Dorrit Aaslyng, Vaerlose (DK)

Correspondence Address: NOVOZYMES NORTH AMERICA, INC., C/O NOVO NORDISK OF NORTH AMERICA, INC., 405 LEXINGTON AVENUE, SUITE 6400, NEW YORK, NY, 10174, US

(73) Assignee: Novozymes A/S, Bagsvaerd (DK)

(21) Appl. No.: 09/899,689

(22) Filed: Jul. 5, 2001

Related U.S. Application Data
(60) Provisional application No. 60/220,538, filed on Jul. 25, 2000.

(30) Foreign Application Priority Data
Jul. 21, 2000 (DK) PA 2000 01121

(51) Int. Cl. C11D 1/00; C09D 9/04; C11D 3/00; C11D 7/18

(52) U.S. Cl. 510/302; 510/205; 510/309; 510/392

(57) ABSTRACT

The invention provides an antimicrobial composition comprising an enzymatic component and one or more non-enzymatic biocides; a method for killing or inhibiting microbial cells comprising a treatment with the antimicrobial composition; and a detergent composition comprising the antimicrobial composition. The invention provides an improved antimicrobial effect.

### CROSS REFERENCE TO RELATED APPLICATIONS

This application claims, under 35 U.S.C. 119, priority or the benefit of Danish application no. PA 2000 01121 filed Jul. 21, 2000 and U.S. application Ser. No. 60/220,538 filed Jul. 25, 2000, the contents of which are fully incorporated herein by reference.

### FIELD OF THE INVENTION

The invention relates to compositions with antimicrobial activity comprising an enzymatic component and one or more non-enzymatic biocides.

### BACKGROUND

Several enzymatic antimicrobial compositions have been disclosed, e.g. WO 99/08531, WO 99/23887 and WO 00/27204. Likewise several biocidal compounds are known in the art.

It is an object of the present invention to provide an antimicrobial composition with improved antimicrobial activity.

### SUMMARY OF THE INVENTION

We have found that the antimicrobial activity of a non-enzymatic biocidal compound is improved when it is combined with an enzymatic component.

According to the present invention there is provided, in a first aspect, an antimicrobial composition comprising an enzymatic component and one or more non-enzymatic biocides.

In a second aspect, the present invention provides a method for killing or inhibiting microbial cells comprising treating said microbial cells with an enzymatic component and one or more non-enzymatic biocides.

In a third aspect, the present invention provides a detergent composition comprising an enzymatic component, one or more non-enzymatic biocides and a surfactant.

The present invention is useful at any locus subject to contamination by bacteria, fungi, yeast or algae; for the preservation of food, beverages, cosmetics, deodorants, contact lens products, food ingredients or enzyme compositions; as a disinfection for use, e.g., on human or animal skin, hair, oral cavity, mucous membranes, wounds, bruises or in the eye; for killing microbial cells in laundry; for incorporation in cleaning compositions; and for disinfection of hard surfaces, in the pulp and paper industry, in the oil industry, or for water treatment.

### DETAILED DESCRIPTION

In the context of the present invention the terms “antimicrobial” and “biocidal” are intended to mean that there is a bactericidal and/or a bacteriostatic and/or fungicidal and/or fungistatic effect and/or a virucidal effect, wherein

The term “bactericidal” is to be understood as capable of killing bacterial cells.

The term “bacteriostatic” is to be understood as capable of inhibiting bacterial growth, i.e. inhibiting growing bacterial cells.

The term “fungicidal” is to be understood as capable of killing fungal cells.

The term “fungistatic” is to be understood as capable of inhibiting fungal growth, i.e. inhibiting growing fungal cells.

The term “virucidal” is to be understood as capable of inactivating virus.

The term “microbial cells” denotes bacterial cells, fungal cells or algae, and the term “microorganism” denotes a fungus (including yeasts) or a bacterium.

In the context of the present invention the term “inhibiting growth of microbial cells” is intended to mean that the cells are in the non-growing state, i.e., that they are not able to propagate.

The term “hard surface” as used herein relates to any surface, which is essentially non-permeable for microorganisms. Examples of hard surfaces are surfaces made from metal, e.g., stainless steel, plastics, rubber, board, glass, wood, paper, textile, concrete, rock, marble, gypsum and ceramic materials which optionally may be coated, e.g., with paint, enamel and the like. The hard surface can also be a process equipment, e.g., a cooling tower, an osmotic membrane, a water treatment plant, a dairy, a food processing plant, a chemical plant, a pharmaceutical process plant, a pulp and paper plant or an oil processing plant. Accordingly, the composition according to the present invention is useful in a conventional cleaning-in-place (C-I-P) system.

Non-enzymatic biocides

In the context of the present invention the term “biocide” includes disinfectants and preservatives, such as bactericides, fungicides, and algaecides.

The term “disinfectant” is defined as a compound which is capable of reducing the number of living cells of Escherichia coli (DSM 1576) to 1/100 after 10 min. incubation at 20° C. in an aqueous solution of 50%(w/w); preferably in an aqueous solution of 40%(w/w); more preferably in an aqueous solution of 25%(w/w); even more preferably in an aqueous solution of 10%(w/w); most preferably in an aqueous solution of 5%(w/w); and in particular in an aqueous solution of 1%(w/w).

The term “preservative” is defined as a compound which is capable of inhibiting the outgrowth of Escherichia coli (DSM1576) for 24 hours at 25° C. in a microbial growth substrate, when added in a concentration of 1000 ppm; preferably when added in a concentration of 500 ppm; more preferably when added in a concentration of 250 ppm; even more preferably when added in a concentration of 100 ppm; most preferably when added in a concentration of 50 ppm; and in particular when added in a concentration of 25 ppm.

The biocides of the composition of the invention may consist of the disinfectants and preservatives defined above.

In an embodiment, the biocide may be a polypeptide having from 2 to 50 amino acid residues, preferably having from 2 to 40 amino acid residues, more preferably having from 2 to 30 amino acid residues, most preferably having from 5 to 30 amino acid residues, and in particular having from 5 to 20 amino acid residues.

In another embodiment, the biocide may not have any enzymatic activity as defined by any enzyme class, such as an enzyme class selected from the group consisting of EC 1. . . , EC 2. . . , EC 3. . . , EC 4. . . , EC 5. . . , and EC 6. . . The biocide may not be a polypeptide having more than 50 amino acid residues; preferably the biocide may not be a polypeptide having more than 30 amino acid residues; more preferably the biocide may not be a polypeptide having more than 10 amino acid residues; and most preferably the biocide is not a polypeptide.

In another embodiment, the biocide is not a substrate for the enzyme(s) included in the composition of the invention. In another embodiment, the biocide is not capable of reacting with the enzyme(s) included in the composition of the invention. In yet another embodiment, the biocide is not a substrate of, or capable of reacting with, an oxidoreductase. In yet another embodiment, the biocide is not a substrate of, or capable of reacting with, a hydrolase as defined in the enzyme class EC 3. . .

The biocides may also be selected from the group consisting of quaternary ammonium compounds, aldehydes, triclosan, organometals, biguanides, phenolics, halogenated organic compounds, inorganics, iodophors and amphoterics.

Preferred biocides are those selected from the group consisting of Benzoic acid (CAS 65-85-0), Sodium benzoate (CAS 532-32-1), Benzylalcohol (CAS 100-51-6), Bronopol (CAS 52-51-7) Chlorhexidine (CAS 55-56-1), Chlorhexidine digluconate (CAS 18472-51-0), Chlorhexidine diacetate (56-95-1), chlorhexidine di-hydrochloride (CAS 3697-42-5), Chloroxylenol (CAS 88-04-0) Dehydroacetic acid (CAS 520-45-6), Sodium dehydroacetate (CAS 4418-26-2), Dichlorobenzyl alcohol (CAS 1777-82-8), Dimethylol di-methyl hydrantoin (CAS 6440-58-0), Ethyl alcohol (CAS 64-17-5), Formaldehyde (CAS 50-00-0), Glutaraldehyde, Imidazolidinyl urea (CAS 39236-46-9) Methylchloroisothiazolinone (CAS 261172-55-4), Benzisothiazolinone, Methylisothiazolinone (CAS 2682-20-4), methylparaben (CAS 99-76-3), ethylparabens (CAS 120-47-8), propylparabens (CA 94-13-3), Butylparabens (CAS 94-26-8), Isopropylparabens (CAS 4191-73-5), Isobutylparaben (CAS 4247-02-3), Benzylparabens (CAS 94-18-8), Phenethyl alcohol (CAS 60-12-8), Phenoxyethanol (CAS 122-99-6), Quaternium-15 (CAS 51229-78-8), Sorbic acid (CAS 110-44 -1) Potassium sorbate (CAS 590-00-1), Dimethyl hydroxymethyl pyrazole (CAS 85264-33-1), lodopropyinyl butylcarbamate (CAS 55406-53-6), Methenammonium chloride (CAS 76902-90-4), Methyldibromo glutaronitrile (CAS 35691-65-7), Polyquaternium-42 (CAS 31075-24-8), Sodium hydroxymethylglycinate (CAS 70161-44-3), Benzalkonium chloride, Benzethonium Chloride (CAS 121 -54-0), 5-Bromo-5-nitro-1,3-dioxane (CAS 30007-47-7), Chloroacetamide (CAS 79-07-2) Chlorobutanol (CAS 57-15-8), Dimethoxane (CAS 828-00-2), Dimethyl Oxazolidine (CAS 51200-87-4), 7-ethyl bicyclooxazolidine (CAS 7747-35-5), Glutaral (CAS 111 -30-8), Hexet 94-6), Phenylmercuric acetate (CAS 62-38-4), Thimersal (CAS 54-64-8), Ort phenylphenol (CAS 90-43-7), Polyaminopropyl biguanide (CAS 27083-27-8), Polymethoxy bicyclic oxazolidi 56709-13-8), Salicylic acid (CAS 69-72-7), Sodium borate (CAS 1303-96-4), Boric acid (CAS 10043-35-3), Sodium iodate (CAS 7681-55-2), Zinc pyrithione (CAS 13463-41-7), Selenium disulfide (CAS 7488-56-4), Piroctone Olamine (CAS 68890-664), Triclosan (CAS 3380-34-5), Triclocarban (CAS 101-20-2), Chloroxylenol, Zinc phenolsulfonate (CAS 127-82-2), essential oils or chelating agents like EDTA (CAS 60-00-4), polyphosphates, Pentetic acids (CAS 67-43-6), Hydroxyethyl ethylenediamine triacetic acid (CAS 150-39-0) and Etidronic acid (CAS 2809-21-4).

Enzymatic component

The enzymatic component comprise one or more enzymes, such as proteases, lipases, cutinases, amylases, carbohydrases, cellulases, pectinases, mannanases, arabinases, galactanases, xylanases and/or oxidoreductases; preferably the enzymatic component comprise at least an oxidoreductase. In an embodiment the enzyme is a hydrolase as defined in the enzyme class EC 3. . .

Oxidoreductases are defined as the enzyme class EC 1. . . Preferred oxidoreductases are phenol oxidizing enzymes, such as oxidases (e.g. laccases) and peroxidases (e.g. haloperoxidases); more preferred oxidoreductases are laccases, peroxidases and haloperoxidases; most preferred oxidoreductases are haloperoxidases. It is to be understood that oxidoreductase variants (e.g. produced by recombinant techniques) are included within the meaning of the term “oxidoreductase”.

In a preferred embodiment the enzymatic component comprise an oxidoreductase and one or more other enzymes, such as proteases, lipases, cutinases, amylases, carbohydrases, cellulases, pectinases, mannanases, arabinases, galactanases and/or xylanases.

In an embodiment, the enzymatic component comprises a lytic enzyme capable of degrading a microbial cell envelope. In another embodiment, the enzymatic component does not comprise a lytic enzyme capable of degrading a microbial cell envelope.

The enzymatic component may comprise compounds known in the art necessary to obtain the desired enzymatic activity, such as oxygen (O₂) in the case of laccases, a source of hydrogen peroxide (H₂O₂) in the case of peroxidases, and a source of halide (chloride, bromide, iodide) in the case of haloperoxidases.

In a preferred embodiment the enzymatic component consists of a haloperoxidase, a source of hydrogen peroxide and a source of halide.

The enzymatic component may also comprise compounds capable of enhancing the enzymatic activity (enhancing agents), and other conventional additives known in the art for stabilizing the enzyme(s), such as polyethylene glycol (PEG) and polymers like polyacrylate or polyvinyl pyrrolidone.

The concentration of the enzyme(s) in the final antimicrobial composition is typically in the range of 0.01-100 ppm, preferably 0.05-50 ppm, more preferably 0.1-20 ppm, and most preferably 0.5-10 ppm.

The enzymatic component may be capable of reducing the number of living cells (killing) of E. coli (DSM1576) to less than 95% (preferably less than 90%, more preferably less than 75%, most preferably less than 50%), when incubated 10 min. at 20° C. in an aqueous solution containing 1 mg/L of the enzymatic component.

The enzymatic component may also be capable of increasing the time before outgrowth (inhibiting) of E. coli (DSM1576) at 25° C. in a microbial growth substrate containing 1 mg/L of the enzymatic component by at least 5%, preferably at least 10%, more preferably at least 25%, and most preferably at least 50%.

Preferred commercially available proteases include Alcalase™, Savinase™, Primase™, Everlase™, Esperase™, and Kannase™ (Novo Nordisk A/S), Maxatase™, Maxacal™, Maxapem™, Properase™, Purafect™, Purafect OxP™, FN2™, FN3™, and FN4™ (Genencor International Inc.).

Preferred commercially available lipase enzymes include Lipolase™, Lipolase Ultra™ and Lipoprime™ (Novo Nordisk A/S).

Preferred commercially available amylases are Duramy|™, Termamy|™, FungaMy|™ and BAN™ (Novo Nordisk A/S), Rapidase™ and Purastar™ (Genencor International Inc.).

Preferred commercially available cellulases include Celluzyme™, and Carezyme™ (Novo Nordisk A/S), Clazinase™, and Puradax HA™ (Genencor International Inc.), and KAC-500(B)™ (Kao Corporation).

Laccases and Compounds possessing Laccase Activity

Compounds possessing laccase activity may be any laccase enzyme comprised by the enzyme classification EC 1.10.3.2, or any fragment derived therefrom, exhibiting laccase activity.

Preferred laccase enzymes and/or laccase related enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinus, e.g., C. cinereus, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radita (WO 92/01046), or Coriolus, e.g., C. hirsutus (JP 2-238885).

Suitable examples from bacteria include a laccase derivable from a strain of Bacillus.

A laccase derived from Coprinus, Myceliophthora, Polyporus, Scytalidium or Rhizoctonia is preferred; in particular a laccase derived from Coprinus cinereus, Myceliophthora thermophila, Polyporus pinsitus, Scytalidium thermophilum or Rhizoctonia solani.

The laccase or the laccase related enzyme may furthermore be one which is producible by a method comprising cultivating a host cell transformed with a recombinant DNA vector which carries a DNA sequence encoding said laccase as well as DNA sequences encoding functions permitting the expression of the DNA sequence encoding the laccase, in a culture medium under conditions permitting the expression of the laccase enzyme, and recovering the laccase from the culture.

Determination of Laccase Activity (LACU)

Laccase activity (particularly suitable for Polyporus laccases) may be determined from the oxidation of syringaldazin under aerobic conditions. The violet colour produced is photometered at 530 nm. The analytical conditions are 19 mM syringaldazin, 23 mM acetate buffer, pH 5.5, 30° C., 1 min. reaction time.

1 laccase unit (LACU) is the amount of enzyme that catalyses the conversion of 1.0 mmole syringaldazin per minute at these conditions.

Determination of Laccase Activity (LAMU)

Laccase activity may be determined from the oxidation of syringaldazin under aerobic conditions. The violet colour produced is measured at 530 nm. The analytical conditions are 19 mM syringaldazin, 23 mM Tris/maleate buffer, pH 7.5, 30° C., 1 min. reaction time.

1 laccase unit (LAMU) is the amount of enzyme that catalyses the conversion of 1.0 mmole syringaldazin per minute at these conditions.

Peroxidases and Compounds possessing Peroxidase Activity

Compounds possessing peroxidase activity may be any peroxidase enzyme comprised by the enzyme classification (EC 1.11.1.7), or any fragment derived therefrom, exhibiting peroxidase activity. In the context of this invention, compounds possessing peroxidase activity comprise peroxidase enzymes and peroxidase active fragments derived from cytochromes, haemoglobin or peroxidase enzymes.

Preferably, the peroxidase employed in the composition of the invention is producible by plants (e.g. horseradish or soybean peroxidase) or microorganisms such as fungi or bacteria.

Some preferred fungi include strains belonging to the subdivision Deuteromycotina, class Hyphomycetes, e.g., Fusarium, Humicola, Trichoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resi

l, Myrothecium verrucaria (IFO 6113), Verticillum alboatrum, Verticillum dahlie, Arthromyces ramosus (FERM P-7754), Caldariomyces fumago, Ulocladium chartarum, Embellisia alli or Dresch/era halodes.

Other preferred fungi include strains belonging to the subdivision Basidiomycotina, class Basidiomycetes, e.g., Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g., T. versicolor (e.g. PR4 28-A).

Further preferred fungi include strains belonging to the subdivision Zygomycotina, class Mycoraceae, e.g., Rhizopus or Mucor, in particular Mucor hiemalis.

Some preferred bacteria include strains of the order Actinomycetales, e.g. Streptomyces spheroides (ATTC 23965), Streptomyces thermoviolaceus (IFO 12382) or Streptoverticillum verticillium ssp. verticillium.

Other preferred bacteria include Bacillus pumilus (ATCC 12905), Bacillus stearothermophilus, Rhodobacter sphaeroides, Rhodomonas palustri, Streptococcus lactis, Pseudomonas purrocinia (ATCC 15958) or Pseudomonas fluorescens (NRRL B-11).

Further preferred bacteria include strains belonging to Myxococcus, e.g., M. virescens.

The peroxidase may furthermore be one which is producible by a method comprising cultivating a host cell transformed with a recombinant DNA vector which carries a DNA sequence encoding said peroxidase as well as DNA sequences encoding functions permitting the expression of the DNA sequence encoding the peroxidase, in a culture medium under conditions permitting the expression of the peroxidase and recovering the peroxidase from the culture.

Particularly, a recombinantly produced peroxidase is a peroxidase derived from a Coprinus sp., in particular C. macrorhizus or C. cinereus according to WO 92/16634.

Determination of Peroxidase Activity (POXU)

One peroxidase unit (POXU) is the amount of enzyme which under the following conditions catalyze the conversion of 1 μmole hydrogen peroxide per minute:

0.1 M phosphate buffer pH 7.0, 0.88 mM hydrogen peroxide, 1.67 mM 2,2′-azino-bis(3-ethylbenzothiazoline-6-sulfonate) (ABTS) and 30°C.

The reaction is followed for 60 seconds (15 seconds after mixing) by the change in absorbance at 418 nm, which should be in the range 0.15 to 0.30.

For calculation of activity is used an absorption coefficient of oxidized ABTS of 36 mM⁻¹ cm⁻¹ and a stoichiometry of one mole H₂O₂ converted per two mole ABTS oxidized.

Haloperoxidases

Haloperoxidases such as chromo-, bromo- and/or iodoperoxidases are suitable enzymes in the composition of the invention. Haloperoxidases form a class of enzymes, which are able to oxidize halides (Cl−, Br−, I−) in the presence of hydrogen peroxide or a hydrogen peroxide generating system to the corresponding hypohalous acids according to:

H₂O₂ +X−+H+−>H₂O +HOX

wherein X—is a halide and HOX is a hypohalous acid.

If a convenient nucleophilic acceptor is present, a reaction will occur with HOX and a halogenated compound will be formed.

There are three types of haloperoxidases, classified according to their specificity for halide ions: Chloroperoxidases (E.C. 1.11.1.10) which catalyse formation of hypo-chlorit from chloride ions, hypo-bromit from bromide ions and hypo-iodit from iodide ions; Bromoperoxidases which catalyse formation of hypo-bromit from bromide ions and hypo-iodit from iodide ions; and iodoperoxidases (E.C. 1.11.1.8) which solely catalyze the formation of hypoiodit from iodide ions. Hypoiodit, however, undergoes spontanous disproportionation to iodine and thus iodine is the observed product. These hypo-halit compounds may subsequently react with other compounds forming halogenated compounds.

Haloperoxidases have been isolated from various organisms: mammals, marine animals, plants, algae, a lichen, fungi and bacteria. It is generally accepted that haloperoxidases are the enzymes responsible for the formation of halogenated compounds in nature, although other enzymes may be involved.

Haloperoxidases have been isolated from many different fungi, in particular from the fungus group dematiaceous hyphomycetes, such as Caldariomyces, e.g., C. fumago, Altemaria, Curvularia, e.g., C. verruculosa and C. inaequalis, Drechslera, Ulocladium and Botrytis.

According to the present invention a haloperoxidase obtainable from Curvularia, in particular C. verruculosa is preferred such as C. verruculosa CBS 147.63 or C. verruculosa CBS 444.70. Curvularia haloperoxidase and recombinant production hereof is described in WO 97/04102.

Haloperoxidases have also been isolated from bacteria such as Pseudomonas, e.g., P. pyrrocinia and Streptomyces, e.g., S. aureofaciens.

In a preferred embodiment the haloperoxidase is derivable from Curvularia sp., in particular C. verruculosa and C. inaequalis.

In a preferred embodiment the haloperoxidase is a vanadium haloperoxidase derivable from a strain of Curvularia inaequalis such as C. inaequalis CBS 102.42 as described in WO 95127046, e.g. a vanadium haloperoxidase encoded by the DNA sequence of WO 95/27046, FIG. 2 all incorporated by reference.

In another preferred embodiment the haloperoxidase is a vanadium haloperoxidase derivable from a strain selected from Drechslera hartlebii, Dendryphiella salina, Phaeotrichoconis crotalarie and Geniculosporium sp. The vanadium haloperoxidase is more preferably derivable from Drechslera hartlebii (DSM 13444), Dendryphielia salina (DSM 13443), Phaeotrichoconis crotalarie (DSM 13441) and Geniculosporium sp. (DSM 13442).

The concentration of the haloperoxidase is typically in the range of 0.01-100 ppm enzyme protein, preferably 0.05-50 ppm enzyme protein, more preferably 0.1-20 ppm enzyme protein, and most preferably 0.5-10 ppm enzyme protein.

Determination of Halo

peroxidase Activity

Microtiter assays are performed by mixing 100 μl of haloperoxidase sample (about 0.2 μg/ml) and 100 μl of 0.3 M sodium phosphate pH 7 buffer—0.5 M potassium bromide—0.008% phenol red, adding the solution to 10 μI of 0.3% H₂O₂, and measuring the absorption at 595 nm as a function of time.

Assays using monochlorodimedone (Sigma M4632, ε=20000 M⁻¹ cm⁻¹ at 290 nm) as a substrate are performed as described below. The decrease in absorption at 290 nm is measured as a function of time. Assays are performed in 0.1 M sodium phosphate or 0.1 M sodium acetate, 50 μM monochlorodimedone, 10 mM KBr/KCI, and 1 mM H₂O₂ using a haloperoxidase concentration of about 1 μg/ml. One HU is defined as 1 micromol of monochlorodimedone chlorinated or brominated per minute at pH 5 and 30° C.

Hydrogen Peroxide Sources

According to the invention the hydrogen peroxide needed for the reaction with the haloperoxidase may be achieved in many different ways: It may be hydrogen peroxide or a hydrogen peroxide precursor, such as, e.g., percarbonate or perborate, or a peroxycarboxylic acid or a salt thereof, or it may be a hydrogen peroxide generating enzyme system, such as, e.g., an oxidase and its substrate. Useful oxidases may be, e.g., a glucose oxidase, a glycerol oxidase or an amino acid oxidase.

It may be advantageous to use enzymatically generated hydrogen peroxide, since this source results in a relatively low concentration of hydrogen peroxide under the biologically relevant conditions. Low concentrations of hydrogen peroxide result in an increase in the rate of haloperoxidase-catalysed reaction.

According to the invention the hydrogen peroxide source needed for the reaction with the haloperoxidase may be added in a concentration corresponding to a hydrogen peroxide concentration in the range of from 0.01-1000 mM, preferably in the range of from 0.1-100 mM.

Halide Sources

According to the invention the halide source needed for the reaction with the haloperoxidase may be achieved in many different ways, e.g., by adding a halide salt: It may be sodium chloride, potassium chloride, sodium bromide, potassium bromide, sodium iodide, or potassium iodide.

The concentration of the halide source will typically correspond to 0.01-1000 mM, preferably in the range of from 0.05-500 mM.

Enhancing Agents

Compounds which, when used in combination with oxidoreductases, are capable of enhancing the antimicrobial effect of the composition of the invention include organic enhancers and inorganic enhancers. Various organic enhancers for various purposes are known in the art (e.g. from WO 94/12620, WO 94/12621, WO 95/01626 and WO 96100179) and may suitably be employed in accordance with the present invention.

One group of preferred organic enhancers is phenolic compounds (alkylsyringates) of the formula:

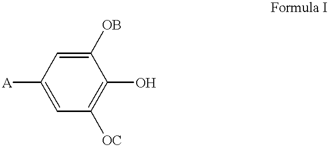

wherein A in said formula denotes a group such as —D, —CH=CH—D, —CH=CH—CH=CH—D, —CH=N—D, —N=N—D, or —N=CH—D, in which D is selected from the group consisting of —CO—E, —SO₂—E, —N—XY, and —N⁺—XYZ, in which E may be —H, —OH, —R, or —OR, and X and Y and Z may be identical or different and selected from —H and —R; R being a C₁—C₁₆ alkyl, preferably a C₁—C₈ alkyl, which alkyl may be saturated or unsaturated, branched or unbranched and optionally substituted with a carboxy, sulpho or amino group; and B and C may be the same or different and selected from CₘH_{2m+1,} where m =1, 2, 3, 4 or 5.

In the above mentioned formula A may be placed meta to the hydroxy group instead of being placed in the para-position as shown.

In particular embodiments of the invention the enhancer is selected from the group having the formula:

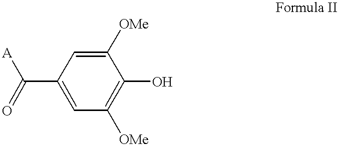

in which A is a group such as —H,—OH,—CH₃,—OCH₃,—O(CH₂)ₙCH₃, where n =1, 2, 3, 4, 5, 6, 7 or 8.

Such enhancers may suitably be present in the composition in an amount between 0.00001-500 mM, preferably 0.0001-5 mM, e.g. 0.001-0.050 mM.

Another preferred group of well performing organic enhancers comprises a —CO—NOH—group and have the following formula:

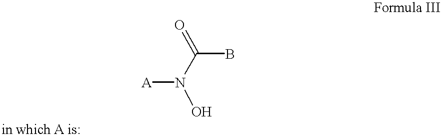

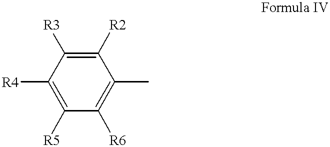

and B is the same as A, or B is H, or C₁—C₁₆ branched or unbranched alkyl wherein said alkyl may contain hydroxy, ether or ester groups, and R2, R3, R4, R5 and R6 are H, OH, NH₂, COOH, SO₃H, C₁C₁₂ branched or unbranched alkyl, acyl, NO₂, CN, Cl, CF₃, NOH-CO-phenyl, C₁—C₆—CO—NOH—A, CO—NOH—A, COR12, phenyl—CO—NOH—A, OR7, NR8R9, COOR10, or NOH—CO—R11, wherein R7, R8, R9, R10 and R11 are C₁-C₁₂ branched or unbranched alkyl or acyl. Within this group of enhancers particularly preferred enhancers are selected from the group consisting of

4-nitrobenzoic acid-N-hydroxyanilide;

4-methoxybenzoic acid-N-hydroxyanilide;

N, N′-dihydroxy-N, N′-diphenylterephthalamide; decanoic acid-N-hydroxyanilide;

N-hydroxy-4-cyanoacetanilide;

N-hydroxy-4-acetylacetanilide;

N-hydroxy-4-hydroxyacetanilide;

N-hydroxy-3-(N′-hydroxyacetamide)acetanilide;

4-cyanobenzoic acid-N-hydroxyanilide;

N-hydroxy-4-nitroacetanilide; and

N-hydroxyacetanilide.

The enhancer may also be one of the compounds disclosed in WO 96/18770 such as N-hydroxy compounds, in particular aliphatic, cycloaliphatic, heterocyclic or aromatic compounds containing NO—, N(OH)—, or N(OH)(R₁), especially N-hydroxy benzotriazol (HOBT), Violuric acid, or N-hydroxyacetanilide (HAA).

In a preferred embodiment of the invention the enhancer is a compound of the general formula (V):

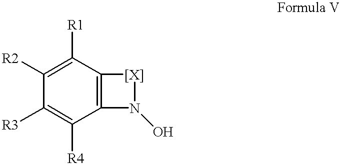

wherein R¹, R², R³, R⁴ are individually selected from the group consisting of hydrogen, halogen, hydroxy, formyl, carboxy and salts and esters thereof, amino, nitro, C₁-C₁₂ alkyl, C₁-C₆ alkoxy, carbonyl(C₁-C₁₂ alkyl), aryl, in particular phenyl, sulpho, aminosulfonyl, carbamoyl, phosphono, phosphonooxy, and salts and esters thereof, wherein the R¹, R², R³, R⁴ may be substituted with R⁵, wherein R⁵ represents hydrogen, halogen, hydroxy, formyl, carboxy and salts and esters thereof, amino, nitro, C₁-C₁₂ alkyl, C₁-C₆ alkoxy, carbonyl(C₁-C₁₂ alkyl), aryl, in particular phenyl, sulpho, aminosulfonyl, carbamoyl, phosphono, phosphonooxy, and salts and esters thereof, [X] represents a group selected from (—N═N—), (—N═CR ⁶—)ₘ, (—CR⁶═N—)ₘ, (—CR⁶═CR⁷—)ₘ, (—CR⁶═N—NR⁷—), (—N═N—CHR⁶—), (—N═CR⁶—NR⁷—), (—N═CR⁶—CHR⁷—), (—CR⁶═N—CHR⁷−), (—CR⁶═CR⁷—NR⁸—), and (—CR⁶═CR⁷—CHR⁸—), wherein R⁶, R⁷, and R⁸ independently of each other are selected from H, OH, NH₂, COOH, SO₃H, C₁₋₆—alkyl, NO₂, CN, Cl, Br, F, CH₂OCH₃, COOCH₃; and m is 1 or 2.

In a more preferred embodiment of the invention the enhancer is a compound of the general formula (VI):

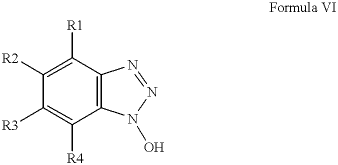

wherein R¹, R², R³, R⁴ are individually selected from the group consisting of hydrogen, halogen, hydroxy, formyl, carboxy and salts and esters thereof, amino, nitro, C₁-C₁₂ alkyl, C₁-C₆ alkoxy, carbonyl(C₁-C₁₂ alkyl), aryl, in particular phenyl, sulpho, aminosulfonyl, carbamoyl, phosphono, phosphonooxy, and salts and esters thereof, wherein the R¹, R², R³, R⁴ may be substituted with R⁵, wherein R⁵ represents hydrogen, halogen, hydroxy, formyl, carboxy and salts and esters thereof, amino, nitro, C₁-C₁₂ alkyl, C₁-C₆ alkoxy, carbonyl(C₁-C₁₂ alkyl), aryl, in particular phenyl, sulpho, aminosulfonyl, carbamoyl, phosphono, phosphonooxy, and salts and esters thereof.

The enhancer may also be a salt or an ester of formula V or VI.

Further preferred enhancers are oxoderivatives and N-hydroxy derivatives of heterocyclic compounds and oximes of oxo- and formyl-derivatives of heterocyclic compounds, said heterocyclic compounds including five-membered nitrogen-containing heterocycles, in particular pyrrol, pyrazole and imidazole and their hydrogenated counterparts (e.g. pyrrolidine) as well as triazoles, such as 1,2,4-triazole; six-membered nitrogen-containing heterocycles, in particular mono-, di- and triazinanes (such as piperidine and piperazine), morpholine and their unsaturated counterparts (e.g. pyridine and pyrimidine); and condensed heterocycles containing the above heterocycles as substructures, e.g. indole, benzothiazole, quinoline and benzoazepine.

Examples of preferred enhancers from these classes of compounds are pyridine aldoximes; N-hydroxypyrrolidinediones such as N-hydroxysuccinimide and N-hydroxyphthalimide; 3,4-dihydro-3-hydroxybenzo[1,2,3]triazine-4-one; formaldoxime trimer (N,N′, N″-trihydroxy-1 ,3,5-triazinane); and violuric acid (1,3-diazinane-2,4,5,6-tetrone-5-oxime).

Still further enhancers which may be applied in the invention include oximes of oxo- and formyl-derivatives of aromatic compounds, such as benzoquinone dioxime and salicylaldoxime (2-hydroxybenzaldehyde oxime), and N-hydroxyamides and N-hydroxyanilides, such as N-hydroxyacetanilide.

Preferred enhancers are selected from the group consisting of 1-hydroxybenzotriazole; 1-hydroxybenzotriazole hydrate; 1-hydroxybenzotriazole sodium salt; 1-hydroxybenzotriazole potassium salt; 1-hydroxybenzotriazole lithium salt; 1-hydroxybenzotriazole ammonium salt; 1-hydroxybenzotriazole calcium salt; 1-hydroxybenzotriazole magnesium salt; and 1-hydroxybenzotriazole-6-sulphonic acid.

A particularly preferred enhancer is 1-hydroxybenzotriazole.

All the specifications of N-hydroxy compounds above are understood to include tautomeric forms such as N-oxides whenever relevant.

In particular, the enhancer of the invention may be the corresponding N-oxyl free radical to any of the compounds disclosed in WO 96/18770 such as TEMPO (2,2,6,6-tetramethylpiperidinoxyl).

The organic enhancers may suitably be present in the paint composition in concentrations from 1 to 1000 μFM, preferably from 5 to 500 μM.

We have observed that an improved haloperoxidase effect may be obtained using an enhancer, preferably an ammonium enhancer, preferably in combination with a halide enhancer or an organic enhancer. The ammonium enhancer may be compounds of the formula:

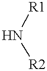

wherein the substituent groups R1 and R2 may be identical or different. R1 and R2 may suitably be any of the following groups: hydrogen, halide, sulphate, phenyl, a straight or branched chain alkyl having from 1 to 14 carbon atoms, or a substituted straight or branched alkyl group having from 1 to 14 carbon atoms where the substituent group is located at C₁-C₁₄ and represent any of the 30 following radicals: hydroxy, halogen, formyl, carboxy, carboxy esters, carboxy salts, carbamoyl, sulfo, sulfo esters, sulfo salts, sulfamoyl, nitro, amino, phenyl, C₁-C₅-alkoxy, carbonyl-C₁-C₅-alkyl, aryl-C₁-C₅-alkyl. Where R1 and/or R2 includes groups selected from carbamoyl, sulfamoyl, and amino groups these groups may furthermore be unsubstituted or substituted once or twice with a substituent group R3, Where R1 and/or R2 includes a phenyl group it may furthermore be unsubstituted or substituted with one or more substituent groups R3. Where R1 and/or R2 includes groups selected from C₁-C₅-alkoxy, carbonyl-C₁-C₅-alkyl, and aryl-C₁-C₅-alkyl these groups may be saturated or unsaturated, branched or unbranched, and may furthermore be unsubstituted or substituted with one or more substituent groups R3. R3 represents any of the following groups: halogen, hydroxy, formyl, carboxy, carboxy esters, carboxy salts, carbamoyl, sulfo, sulfo esters, sulfo salts, sulfamoyl, nitro, amino, phenyl, aminoalkyl, piperidino, piperazinyl, pyrrolidin-1-yl, C₁-C₅-alkyl, C₁-C₅-alkoxy. Where R3 includes groups selected from carbamoyl, sulfamoyl, and amino these groups may furthermore be unsubstituted or substituted once or twice with hydroxy, C₁-C₅-alkyl, C₁-C₅-alkoxy. Where R3 includes phenyl this group may furthermore be substituted with one or more of the following groups: halogen, hydroxy, amino,

r formyl, carboxy, carboxy esters, carboxy salts, carbamoyl, sulfo, sulfo esters, sulfo salts, and sulfamoyl. Where R3 includes groups selected from C₁-C₅-alkyl, and C₁-C₅-alkoxy these groups may furthermore be saturated or unsaturated, branched or unbranched, and may furthermore be substituted once or twice with any of the following radicals: halogen, hydroxy, amino, formyl, carboxy, carboxy esters, carboxy salts, carbamoyl, sulfo, sulfo esters, sulfo salts, and sulfamoyl. R1 and R2 may also suitably together a group -B-, in which B represents any of the following groups: (—CHR3—N═N—), (—CH═CH—)ₙ or (—CH═N—)ₙ in which groups n-represents an integer of from 1 to 3 and R3 is a substituent group as defined, supra. (It is to be understood that if the above mentioned formula comprises two or more R3-substituent groups, these R3-substituent groups may be the same or different).

As used herein, the ammonium enhancer may be in their cationic form.

In a preferred embodiment R1 is hydrogen.

In another preferred embodiment R1 is hydrogen and R2 is an alcohol (amino alcohol), e.g., ethanol amine.

In a further preferred embodiment the ammonium enhancer is an ammonium salt, i.e. any ammonium salt known in the art: e.g., diammonium sulphate, ammonium chloride, ammonium bromide, or ammonium iodide.

The ammonium enhancer may suitably be present in the paint composition of the invention in a concentration corresponding to an ammonium concentration in the range of from 0.01-1000 mM, preferably in the range of from 0.05-500 mM.

Composition

The present invention provides an antimicrobial composition, comprising an enzymatic component and one or more non-enzymatic biocides.

The enzymatic component and the non-enzymatic biocides of the composition may be selected so that a synergistic antimicrobial effect is obtained.

The enzymatic component and the non-enzymatic biocides of the composition may be selected so that the number of living cells of E. coli (DSM1576), when incubated 10 min. at 20° C. in an aqueous solution containing 50% w/w (preferably 25% w/w, more preferably 10% w/w, most preferably 5% w/w) of the biocide and 0.5 ppm (preferably 0.1 ppm) of the enzymatic 10 component, are reduced at least 5% (preferably at least 10%) more than compared to what is obtained by adding the results of separate incubations with the biocides and the enzymatic component alone, i.e. a simple additive effect.

The enzymatic component and the non-enzymatic biocides of the composition may also be selected so that the outgrowth of E. coli (DSM1576) at 25° C. in a microbial growth substrate containing 500 ppm (preferably 250 ppm, more preferably 100 ppm, most preferably 50 ppm) of the biocide and 0.5 ppm (preferably 0.1 ppm) of the enzymatic component, are inhibited at least 5% (preferably at least 10%) longer time than compared to what is obtained by adding the results of separate incubations with the biocides and the enzymatic component alone, i.e. a simple additive effect.

The composition may be formulated as a solid, liquid, gel or paste.

When formulated as a solid all components may be mixed together, e.g., as a powder, a granulate or a gelled product.

When other than dry form compositions are used and even in that case, it is preferred to use a two-part formulation system having the enzyme(s) separate from the rest of the composition.

The composition of the invention may further comprise auxiliary agents such as wetting agents, thickening agents, buffer, stabilisers, perfume, colourants, fillers and the like.

Useful wetting agents are surfactants, i.e., non-ionic, anionic, amphoteric or zwitterionic surfactants.

The composition of the invention may be a concentrated product or a ready-to-use product. 30 In use, the concentrated product is typically diluted with water to provide a medium having an effective antimicrobial activity, applied to the object to be disinfected or preserved, and allowed to react with the microorganisms present.

The pH of an aqueous solution of the composition is in the range of from pH 2 to 11, preferably in the range of from pH 4 to 10, more preferably in the range of from pH 5 to 9, and most preferably in the range of from pH 6 to 8.

Method

The present invention provides a method for killing or inhibiting microbial cells comprising treating said microbial cells with the antimicrobial composition of the invention.

The microbial cells may be treated with the enzymatic component and the non-enzymatic biocides simultaneously, in sequential treatments or even in discrete treatments separated by other process steps.

Uses

The invention also encompasses various uses of a composition comprising an enzymatic component and one or more non-enzymatic biocides. Said composition is typically useful at any locus subject to contamination by bacteria, fungi, yeast or algae. Typically, loci are in aqueous systems such as cooling water systems, laundry rinse water, oil systems such as cutting oils, lubricants, oil fields and the like, where microorganisms need to be killed or where their growth needs to be controlled. However, the present invention may also be used in all applications for which known antimicrobial compositions are useful, such as protection of wood, latex, adhesive, glue, paper, cardboard, textile, leather, plastics, caulking, and feed.

Other uses include preservation of foods, beverages, cosmetics such as lotions, creams, gels, ointments, soaps, shampoos, conditioners, antiperspirants, deodorants, mouth wash, contact lens products, enzyme formulations, or food ingredients.

Thus, the composition used in the method of the invention may by useful as a disinfectant, e.g., in the treatment of acne, infections in the eye or the mouth, skin infections; in antiperspirants or deodorants; in foot bath salts; for cleaning and disinfection of contact lenses, hard surfaces, teeth (oral care), wounds, bruises and the like.

In general it is contemplated that the composition of the present invention is useful for cleaning, disinfecting or inhibiting microbial growth on any hard surface. Examples of surfaces, which may advantageously be contacted with the composition of the invention are surfaces of process equipment used e.g. dairies, chemical or pharmaceutical process plants, water sanitation systems, oil processing plants, paper pulp processing plants, water treatment plants, and cooling towers. The composition of the invention should be used in an amount, which is effective for cleaning, disinfecting or inhibiting microbial growth on the surface in question.

Further, it is contemplated that the composition of the invention can advantageously be used in a cleaning-in-place (C.I.P.) system for cleaning of process equipment of any kind.

The method of the invention may additionally be used for cleaning surfaces and cooking utensils in food processing plants and in any area in which food is prepared or served such as hospitals, nursing homes, restaurants, especially fast food restaurants, delicatessens and the like. It may also be used as an antimicrobial in food products and would be especially useful as a surface antimicrobial in cheeses, fruits and vegetables and food on salad bars.

It may also be used as a preservation agent or a disinfection agent in water based paints.

The composition of the present invention is also useful for microbial control of water lines, and for disinfection of water, in particular for disinfection of industrial water.

Detergent composition

The antimicrobial composition of the invention may be added to and thus become a component of a detergent composition.

The detergent composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

In a specific aspect, the invention provides a detergent additive comprising the antimicrobial composition of the invention and a surfactant. The detergent additive as well as the detergent composition may comprise one or more other enzymes such as a protease, a lipase, a cutinase, an amylase, a carbohydrase, a cellulase, a pectinase, a mannanase, an arabinase, a galactanase, a xylanase, an oxidase, e.g., a laccase, and/or a peroxidase.

In general the properties of the chosen enzyme(s) should be compatible with the selected detergent, (i.e. pH-optimum, compatibility with other enzymatic and non-enzymatic ingredients, etc.), and the enzyme(s) should be present in effective amounts. Proteases: Suitable proteases include those of animal, vegetable or microbial origin. Microbial origin is preferred. Chemically modified or protein engineered mutants are included. The protease may be a serine protease or a metallo protease, preferably an alkaline microbial protease or a trypsin-like protease. Examples of alkaline proteases are subtilisins, especially those derived from Bacillus, e.g., subtilisin Novo, subtilisin Carlsberg, subtilisin 309, subtilisin 147 and subtilisin 168 (described in WO 89/06279). Examples of trypsin-like proteases are trypsin (e.g. of porcine or bovine origin) and the Fusarium protease described in WO 89/06270 and WO 94/25583.

Examples of useful proteases are the variants described in WO 92/19729, WO 98/20115, WO 98/20116, and WO 98/34946, especially the variants with substitutions in one or more of the following positions: 27, 36, 57, 76, 87, 97, 101, 104, 120, 123, 167, 170, 194, 206, 218, 222, 224, 235 and 274.

Preferred commercially available protease enzymes include Alcalase™, Savinase™, Primase™, Duralase™, Esperase™, and Kannase™ (Novo Nordisk A/S), Maxatase™, Maxacal™, Maxapem™, Properase™, Purafect™, Purafect OxP™, FN2™, and FN3™ (Genencor International Inc.). Lipases: Suitable lipases include those of bacterial or fungal origin. Chemically modified or protein engineered mutants are included. Examples of useful lipases include lipases from Humicola (synonym Thermomyces), e.g. from H. lanuginosa (T. lanuginosus) as described in EP 258 068 and EP 305 216 or from H. insolens as described in WO 96/13580, a Pseudomonas lipase, e.g. from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g. from B. subtilis (Dartois et al. (1993), Biochemica et Biophysica Acta, 1131, 253-360), B. stearothermophilus (JP 64/744992) or B. pumilus (WO 91/16422). Other examples are lipase variants such as those described in WO 92/05249, WO 94/01541, EP 407 225, EP 260 105, WO 95/35381, WO 96/00292, WO 95/30744, WO 94/25578, WO 95/14783, WO 95122615, WO 97/04079 and WO 97/07202.

Preferred commercially available lipase enzymes include Lipolase™, Lipolase Ultra™ and Lipoprime™ (Novo Nordisk A/S). Amylases: Suitable amylases (a and/or b) include those of bacterial or fungal origin. Chemically modified or protein engineered mutants are included. Amylases include, for example, a-amylases obtained from Bacillus, e.g. a special strain of B. licheniformis, described in more detail in GB 1,296,839.

Examples of useful amylases are the variants described in WO 94/02597, WO 94/18314, WO 96/23873, and WO 97/43424, especially the variants with substitutions in one or more of the following positions: 15, 23, 105, 106, 124, 128, 133, 154, 156, 181, 188, 190, 197, 202, 208, 209, 243, 264, 304, 305, 391, 408, and 444.

Commercially available amylases are Duramy|™, Termamy|™, Fungamy|™ and BAN™(Novo Nordisk A/S), Rapidase™ and Purastar™ (Genencor International Inc.). Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g. the fungal cellulases produced from Humicola insolens, Myceliophthora thermophila and Fusarium oxysporum disclosed in U.S. Pat. No. 4,435,307, U.S. Pat. No. 5,648,263, U.S. Pat. No. 5,691,178, U.S. Pat. No. 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. No. 5,457,046, U.S. No. 5,686,593, U.S. Pat. No. 5,763,254, WO 95/24471, WO 98/12307 and PCT/DK98/00299.

Commercially available cellulases include Celluzyme™, and Carezyme™ (Novo Nordisk A/S), Clazinase™, and Puradax HA™ (Genencor International Inc.), and KAC-500(B)™ (Kao Corporation). Peroxidases/Oxidases: Suitable peroxidases/oxidases include those of plant, bacterial or fungal origin. Chemically modified or protein engineered mutants are included. Examples of useful peroxidases include peroxidases from Coprinus, e.g. from C. cinereus, and variants thereof as those described in WO 93/24618, WO 95/10602, and WO 98/15257.

Commercially available peroxidases include Guardzyme™ (Novo Nordisk A/S).

The detergent enzyme(s) may be included in a detergent composition by adding separate additives containing one or more enzymes, or by adding a combined additive comprising all of these enzymes. A detergent additive of the invention, i.e. a separate additive or a combined additive, can be formulated e.g. as a granulate, a liquid, a slurry, etc. Preferred detergent additive formulations are granulates, in particular non-dusting granulates, liquids, in particular stabilized liquids, or slurries.

Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP 238,216.

The detergent composition of the invention may be in any convenient form, e.g., a bar, a ablet, a powder, a granule, a paste or a liquid. A liquid detergent may be aqueous, typically containing up to 70% water and 0-30% organic solvent, or non-aqueous.

The detergent composition comprises one or more surfactants, which may be non-ionic including semi-polar and/or anionic and/or cationic and/or zwitterionic. The surfactants are typically present at a level of from 0.1% to 60% by weight.

When included therein the detergent will usually contain from about 1% to about 40% of an anionic surfactant such as linear alkylbenzenesulfonate, alpha-olefinsulfonate, alkyl sulfate (fatty alcohol sulfate), alcohol ethoxysulfate, secondary alkanesulfonate, alpha-sulfo fatty acid methyl ester, alkyl- or alkenylsuccinic acid or soap.

When included therein the detergent will usually contain from about 0.2% to about 40% of a non-ionic surfactant such as alcohol ethoxylate, nonylphenol ethoxylate, alkylpolyglycoside, alkyldimethylamineoxide, ethoxylated fatty acid monoethanolamide, fatty acid monoethanolamide, polyhydroxy alkyl fatty acid amide, or N-acyl N-alkyl derivatives of glucosamine (“glucamides”).

The detergent may contain 0-65 % of a detergent builder or complexing agent such as zeolite, diphosphate, triphosphate, phosphonate, carbonate, citrate, nitrilotriacetic acid, ethylenediaminetetraacetic acid, diethylenetriaminepentaacetic acid, alkyl- or alkenylsuccinic acid, soluble silicates or layered silicates (e.g. SKS-6 from Hoechst).

The detergent may comprise one or more polymers. Examples are carboxymethylcellulose, poly(vinylpyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid copolymers.

The detergent may contain a bleaching system which may comprise a H202 source such as perborate or percarbonate which may be combined with a peracid-forming bleach activator such as tetraacetylethylenediamine or nonanoyloxybenzenesulfonate. Alternatively, the bleaching system may comprise peroxyacids of e.g. the amide, imide, or sulfone type.

The enzyme(s) of the detergent composition of the invention may be stabilized using conventional stabilizing agents, e.g., a polyol such as propylene glycol or glycerol, a sugar or sugar alcohol, lactic acid, boric acid, or a boric acid derivative, e.g., an aromatic borate ester, or a phenyl boronic acid derivative such as 4-formylphenyl boronic acid, and the composition may be formulated as described in e.g. WO 92/19709 and WO 92/19708.

The detergent may also contain other conventional detergent ingredients such as e.g. fabric conditioners including clays, foam boosters, suds suppressors, anti-corrosion agents, soil-suspending agents, anti-soil redeposition agents, dyes, bactericides, optical brighteners, hydrotropes, tarnish inhibitors, or perfumes.

It is at present contemplated that in the detergent compositions any enzyme, in particular the haloperoxidase of the invention, may be added in an amount corresponding to 0.01-100 mg of enzyme protein per liter of wash liquor, preferably 0.05-10 mg of enzyme protein per liter of wash liqour, more preferably 0.1-5 mg of enzyme protein per liter of wash liquor, and most preferably 0.1 -1 mg of enzyme protein per liter of wash liquor.

The antimicrobial composition of the invention may additionally be incorporated in the detergent formulations disclosed in WO 97/07202 which is hereby incorporated as reference.

The present invention is further illustrated in the following examples, which are not in any way intended to limit the scope of the invention as claimed.

Materials and Methods

The Malthus Flexi M2060 instrument is available from Malthus Instruments Limited, England.

The Curvularia verruculosa recombinant peroxidase is available from Novo Nordisk A/S, Denmark.

NOPA V0054 powder detergent is available from Nordisk Detergent A/S, Denmark.

Brain Heart Infusion Broth (#CM225) and Tryptone Soya Agar (#CM129) is available from Oxoid, England.

The buffers (0.0005 M) used are:

pH 5: Homopipes (#6047H, Research Organics, U.S.)

pH 6: MES (#M2250, Sigma)

pH 7: HEPES (#H3375, Sigma)

pH 8: HEPES (#H3375, Sigma)

pH 9: HEPES (#H3375, Sigma) +CAPS (#C2632, Sigma)

pH 10: CAPS (#C2632, Sigma) CFU/ml: Colony Forming Units per ml.

Determination of Antimicrobial Activity

Antimicrobial activity may be measured in terms of the number of log reductions. The term “log reduction” is defined as a logarithmic reduction of the number of living cells, e.g. 1 log reduction corresponds to a reduction in living cell number of Escherichia coli DSM1576 or Enterococcus faecalis DSM2570 from Y ×10^{×}CFU/M (CFU: Colony Forming Units, M: ml or g) to Y ×10^{×-1} CFU/M, where X can be 1, 2, 3, 4, 5, 6, 7, 8, 9, 10 or 11, and Y can be any number from 0 to 10. The number of living bacteria are to be determined as the number of E. coli or E. aecalis, respectively, which can grow on Tryptone Soya Agar plates at 30° C.

### EXAMPLE 1

Antibacterial activity of Curvularia verruculosa recombinant haloperoxidase and methylparaben, ethylparaben

, methvlchloroisothiazolinone or benzisothiazolinone

The antibacterial activity of recombinant Curvularia verruculosa haloperoxidase (0.5 and 1 mg/I) and methylparaben (50 and 500 ppm), ethylparaben (50 and 500 ppm), methylchloroisothiazolinone (15 and 30 ppm) or benzisothiazolinone (50 and 500 ppm) is tested against Staphylococcus epidermidis DSM20042 with KBr (5 mM) as halide, (NH₄)₂SO₄ (0 and 5 mM) as enhancing agent, and hydrogen peroxide (0.5 mM) at pH 6-8. S. epidermidis is grown in Brain Heart Infusion Broth (BHI) at 30° C. and diluted in the buffers, respectively to a concentration of approximately 10⁶ CFU/ml. The cell suspensions are incubated with the enzyme/biocide system for 15 min at 40° C.

The bactericidal activity is determined by incubation in a Malthus instrument. The detection times measured by the Malthus instrument are converted to CFU/ml by a calibration curve. Either direct or indirect Malthus measurements are used when enumerating total survival cells. By the direct measurements, the cell metabolism is determined by conductance measurements in the growth substrate. By the indirect measurements, 3 ml of growth medium is transferred to the outer chamber of the indirect Malthus cells, and 0.5 ml of sterile KOH (0.1 M) is transferred to the inner chamber. The cell suspensions are after enzyme treatment transferred to the outer chamber of the Malthus cell. As cells are growing in the outer chamber they produce CO₂ which will dissolve in the KOH in the inner chamber and thereby change the conductance of the KOH. The amount of CO₂ formed by the respiring cells surviving the enzyme treatment is used for estimating the number of viable cells. When the conductance change is measurable by the Malthus instrument, a detection time (dt) will be recorded. The dt's are converted to colony counts by use of a calibration curve relating CFU/ml to dt.

### EXAMPLE 2

Antibacterial activity in detergent of haloperoxidase and biocide

The antibacterial activity of Curvularia verruculosa haloperoxidase (0.5 mg/I) and methylparaben (0, 50 and 500 ppm), ethylparaben (0, 50 and 500 ppm), methylchloroisothiazolinone (0, 15 and 30 ppm) or benzisothiazolinone (0, 50 and 500 ppm) is tested in NOPA V0054 powder detergent. pH of the detergent is measured as approximately 9.9, antimicrobial activity is evaluated in the detergent at pH 9.9, 9, and 8 where pH is adjusted. Antimicrobial activity of the enzyme/biocide system is determined using KBr (2 and 4 mM) as halide, (NH₄)₂SO₄ (0 and 2 mM) as enhancing agent, and H₂O₂ (0.5 mM) as oxidizing agent.

Microbial cells (Escherichia coli DSM1576) are grown over night in Tryptone Soy Broth, this strain is not found to be sensitive to the detergent when no enzyme/biocide system is present. Cells are suspended in NOPA detergent (6 g/L) to the cell concentration of approximately 10⁷-10⁸ CFU/mI, followed by addition of the enzyme/biocide system. After incubation at 35° C. for 12 min, the number of living microorganisms was determined by use of a Malthus instrument.

1. An antimicrobial composition comprising an enzymatic component and one or more non-enzymatic biocides.

2. The composition of claim 1, wherein the enzymatic component comprises an oxidoreductase and a suitable oxidizing agent.

3. The composition of claim 2, wherein the enzymatic component comprises a peroxidase and a source of hydrogen peroxide.

4. The composition of claim 2, wherein the enzymatic component comprises a haloperoxidase, a source of hydrogen peroxide and a source of halide.

5. The composition of claim 2, wherein the enzymatic component comprises a laccase and a source of oxygen.

6. The composition of claim 1, which further comprises one or more enzymes selected from the group consisting of proteases, lipases, cutinases, amylases, carbohydrases, cellulases, pectinases, mannanases, arabinases, galactanases and xylanases.

7. The composition of claim 1, wherein the non-enzymatic biocides are capable of either:
(a) reducing the number of living cells of Escherichia coli (DSM 1576) to 1/100 after 10 min. incubation at 20° C. in an aqueous solution of 50%(w/w), or
(b) inhibiting the outgrowth of Escherichia coli (DSM1576) for 24 hours at 25° C. in a microbial growth substrate, when added in a concentration of 1000 ppm.

8. The composition of claim 1, wherein the non-enzymatic biocides are selected from the group consisting of Benzoic acid, Sodium benzoate, benzylalcohol, Bronopol, Chlorhexidine, Chlorhexidine digluconate, Chlorhexidine diacetate, chlorhexidine di-hydrochloride, Chloroxylenol, Dehydroacetic acid, Sodium dehydroacetate, Dichlorobenzyl alcohol, Dimethylol di-methyl hydrantoin, Ethyl alcohol, Formaldehyde, Glutaraldehyde, Imidazolidinyl urea, Methylchloroisothiazolinone, Benzisothiazolinone, Methylisothiazolinone, methylparaben, ethylparabens, propylparabens, Butylparabens, Isopropylparabens, Isobutylparabens, Benzylparabens, Phenethyl alcohol, Phenoxyethanol, Quaternium-15, Sorbic acid, Potassium sorbate, Dimethyl hydroxymethyl pyrazole, lodopropyinyl butylcarbamate, Methenammonium chloride, Methyldibromo glutaronitrile, Polyquaternium-42, Sodium hydroxymethylglycinate, Benzalkonium chloride, Benzethonium Chloride, 5-Bromo-5-nitro-1,3-dioxane, Chloroacetamide, Chlorobutanol, Dimethoxane, Dimethyl Oxazolidine, 7-ethyl bicyclooxazolidine, Glutaral, Hexetidine, Phenylmercuric acetate, Thimersal, Ortho phenylphenol, Polyaminopropyl biguanide, Polymethoxy bicyclic oxazolidine, Salicylic acid, Sodium borate, Boric acid, Sodium iodate, Zinc pyrithione, Selenium disulfide, Piroctone Olamine, Triclosan, Triclocarban, Chloroxylenol, Zinc phenolsulfonate, essential oils or chelating agents like EDTA, polyphosphates, Pentetic acids, Hydroxyethyl ethylenediamine triacetic acid and Etidronic acid.

9. A method for killing or inhibiting microbial cells, comprising treating the microbial cells with the composition of claim 1.

10. A method for cleaning, disinfecting or inhibiting microbial growth on a hard surface, comprising contacting the hard surface with the composition of claim 1.

11. The method of claim 10, wherein the hard surface is a process equipment

12. The method of claim 11, wherein the hard surface is a member of a cooling tower, a water treatment plant, a dairy, a food processing plant, a chemical or pharmaceutical process plant.

13. The method of claim 11, wherein the hard surface is a surface of water sanitation equipment.

14. The method of claim 11, wherein the hard surface is a surface of equipment for pulp and paper processing.

15. A method for preserving a cosmetic product, comprising adding an effective amount of the composition of claim 1 into the cosmetic product.

16. The method of claim 15, wherein the cosmetic product is selected from the group consisting of a mouth wash composition, a cosmetic liquid or gel or paste, an eye lotion, a perspirant, a deodorant, a nasal spray, an eye ointment, an ointment or cream and a foot bath salt.

17. A detergent composition comprising an enzymatic component, one or more non-enzymatic biocides, and a surfactant.

18. A detergent composition comprising a surfactant and the composition of claim 2.
